(19) United States
(12) Patent Application Publication
(10) Pub. No.: US 2023/0210677 A1
(43) Pub. Date: Jul. 6, 2023

(54) ANTI-MIGRATION STENT

(71) Applicant: BOSTON SCIENTIFIC SCIMED, INC., Maple Grove, MN (US)

(72) Inventors: Paul Smith, Smithfield, RI (US); Shawn Ryan, Littleton, MA (US)

(73) Assignee: BOSTON SCIENTIFIC SCIMED, INC., Maple Grove, MN (US)

(21) Appl. No.: 18/093,233

(22) Filed: Jan. 4, 2023

Related U.S. Application Data
(60) Provisional application No. 63/296,563, filed on Jan. 5, 2022.

(51) Int. Cl. A61F 2/848

(52) U.S. Cl. CPC A61F 2/848

(57) ABSTRACT

A system for implantation of a stent in a body lumen may include an elongate tubular member having a lumen extending therein, a stent configured to shift from a delivery configuration to a deployed configuration, and an adhesive structure configured to secure the stent to the body lumen in the deployed configuration. The stent may be disposed within the lumen of the elongate tubular member in the delivery configuration. The adhesive structure may be disposed within the lumen of the elongate tubular member adjacent the stent in a substantially inert state. The adhesive structure may be configured to adhere to the stent and the body lumen after the stent and the adhesive structure are deployed into the body lumen from the lumen of the elongate tubular member.

### CROSS-REFERENCE TO RELATED APPLICATIONS

This application claims the benefit of U.S. Provisional Patent Application Ser. No. 63/296,563, filed Jan. 5, 2022, which is incorporated herein by reference.

### TECHNICAL FIELD

The present disclosure pertains to medical devices, and methods for manufacturing and/or using medical devices. More particularly, the present disclosure pertains to an improved design for an endoprosthesis or stent.

### BACKGROUND

Stents, grafts, stent-grafts, and similar implantable medical devices, collectively referred to hereinafter as stents, are radially expandable or self-expanding endoprostheses which are intravascular or endoscopic implants capable of being implanted transluminally either percutaneously or endoscopically. Stents may be implanted in a variety of body lumens or vessels such as within the vascular system, urinary tracts, bile ducts, gastro-intestinal tract, airways, etc. Stents may be used to open constricted body lumens. Stents may be used to reinforce body vessels and to prevent restenosis following angioplasty in the vascular system. They may be self-expanding, mechanically expandable, or hybrid expandable. In general, self-expanding stents are mounted on a delivery device consisting of two tubes. The stent is delivered by sliding the outer tube to uncover and release the stent.

Stents are typically tubular members that are radially expandable from a reduced diameter configuration for delivery through a patient's body lumen to an expanded configuration once deployed at the treatment site. The stent may be formed from a tubular member in which a pattern is subsequently formed by etching or cutting material from the tubular member, or it may be made from wires or filaments using techniques such as braiding, knitting, or weaving. Desirable stent properties include sufficient flexibility to be able to conform to the tortuous body lumen during delivery, yet sufficient rigidity to resist migration once deployed at the treatment site.

In some stents, the compressible and flexible properties that assist in stent delivery may also result in a stent that tends to migrate from its originally deployed position. Stent migration affects many endoscopic stents including esophageal, duodenal, colonic, pancreatic, biliary and airway stents. It is thus desirable to provide a stent configuration that resists migration following deployment.

Some techniques that have been developed to prevent stent migration including adding barbs and flares to the stent itself or using clips or sutures to attach the stent to the vessel wall. However, there remains a need for an improved stent that is resistant to migration.

### SUMMARY

In one example, a system for implantation of a stent in a body lumen may comprise an elongate tubular member having a lumen extending therein, a stent configured to shift from a delivery configuration to a deployed configuration, and an adhesive structure configured to secure the stent to the body lumen in the deployed configuration. The stent may be disposed within the lumen of the elongate tubular member in the delivery configuration. The adhesive structure may be disposed within the lumen of the elongate tubular member adjacent the stent in a substantially inert state. The adhesive structure may be configured to adhere to the stent and the body lumen after the stent and the adhesive structure are deployed into the body lumen from the lumen of the elongate tubular member.

In addition or alternatively to any example described herein, the adhesive structure is unsecured to the stent within the lumen of the elongate tubular member.

In addition or alternatively to any example described herein, the adhesive structure includes an adhesive element that is activated upon deployment within the body lumen.

In addition or alternatively to any example described herein, the adhesive element is pressure sensitive.

In addition or alternatively to any example described herein, the adhesive structure is disposed radially outward of the stent within the lumen of the elongate tubular member.

In addition or alternatively to any example described herein, after the stent and the adhesive structure are deployed into the body lumen, the stent exerts a radially outward force against the adhesive structure and the body lumen.

In addition or alternatively to any example described herein, the radially outward force increases adhesion between the stent and the adhesive structure and between the adhesive structure and the body lumen.

In addition or alternatively to any example described herein, the adhesive structure extends circumferentially around a majority of a circumference of the stent in the delivery configuration.

In addition or alternatively to any example described herein, in the substantially inert state the adhesive structure has a thickness between about 0.254 mm and about 2.032 mm.

In addition or alternatively to any example described herein, a system for implantation of a stent in a body lumen may comprise an elongate tubular member having a lumen extending therein, a stent configured to shift from a delivery configuration to a deployed configuration, and an adhesive structure configured to secure the stent to the body lumen in the deployed configuration. The stent may be disposed within the lumen of the elongate tubular member in the delivery configuration. The adhesive structure may be disposed within the lumen of the elongate tubular member adjacent the stent in a substantially inert state. The adhesive structure may be configured to adhere to the stent and the body lumen after the stent and the adhesive structure are deployed into the body lumen from the lumen of the elongate tubular member. The adhesive structure may be disposed radially inward of the stent within the lumen of the elongate tubular member.

In addition or alternatively to any example described herein, after the stent and the adhesive structure are deployed into the body lumen from the lumen of the elongate tubular member, the adhesive structure is configured to adhere to an inner surface of the stent and the adhesive structure is configured to adhere to the body lumen through interstices formed in the stent.

In addition or alternatively to any example described herein, the adhesive structure includes a cross-linked polymer.

In addition or alternatively to any example described herein, the adhesive structure includes a plurality of interwoven fibers defining a patch.

In addition or alternatively to any example described herein, the adhesive structure extends longitudinally beyond at least one end of the stent.

In addition or alternatively to any example described herein, a method of securing a stent within a body lumen may comprise advancing an elongate tubular member to a target site within the body lumen, the elongate tubular member having a stent and an adhesive structure retained therein; exposing the adhesive structure to the target site; deploying the stent at the target site to move the adhesive structure into contact with the target site; and applying radially outward force to the adhesive structure for a predetermined period of time sufficient to adhere the adhesive structure to the stent and the target site.

In addition or alternatively to any example described herein, an elongate inner member is disposed radially inward of the elongate tubular member to define an annular space, the stent and the adhesive structure being disposed within the annular space.

In addition or alternatively to any example described herein, the method may further comprise injecting a fluid into the annular space as the adhesive structure is being exposed to the target site to wet the adhesive structure.

In addition or alternatively to any example described herein, the adhesive structure includes an adhesive element and wetting the adhesive structure activates the adhesive element.

In addition or alternatively to any example described herein, applying radially outward force to the adhesive structure at least partially embeds the stent within the adhesive structure.

In addition or alternatively to any example described herein, the adhesive structure is detached from the stent within the elongate tubular member.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a system for implantation of a stent in a body lumen;

FIG. 2 illustrates a cross-section of a portion of the system of FIG. 1;

FIG. 3 illustrates selected aspects related to the implantation of the stent of FIG. 1 in the body lumen;

FIG. 4 illustrates selected aspects of a system for implantation of a stent in a body lumen;

FIG. 5 illustrates a cross-section of a portion of the system of FIG. 4;

FIG. 6 illustrates selected aspects related to the implantation of the stent of FIG. 3 in the body lumen;

FIG. 7 is a cross-sectional view illustrating selected aspects of a stent according to the disclosure implanted in the body lumen;

FIG. 8 illustrates selected aspects of an alternative configuration of the system and/or stent; and

FIG. 9 illustrates selected aspects of an alternative configuration of the system and/or stent.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate exemplary aspects of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, all elements of the disclosure are not necessarily shown in each figure or discussed in detail below. However, it will be understood that the following discussion may apply equally to any and/or all of the components for which there are more than one, unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures illustrate selected components and/or arrangements of an endoprosthesis or stent. It should be noted that in any given figure, some features of the endoprosthesis or stent may not be shown, or may be shown schematically, for simplicity. Additional details regarding some of the components of the endoprosthesis or stent may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the filament”, “the cell”, “the strut”, or other features may be equally referred to all instances and quantities beyond one of said feature. As such, it will be understood that the following discussion may apply equally to any and/or all of the components for which there are more than one within the endoprosthesis or stent, unless explicitly stated to the contrary. Additionally, not all instances of some elements or features may be shown in each figure for clarity.

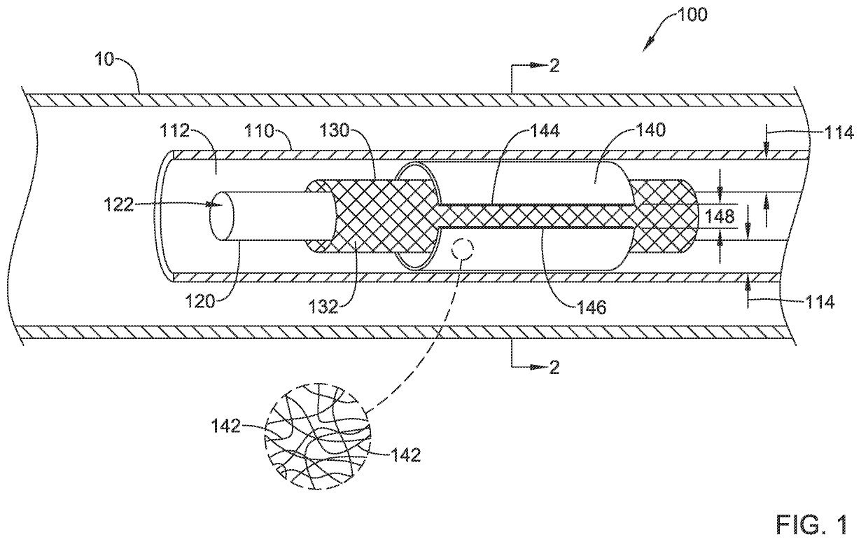

FIGS. 1-3 illustrate selected aspects of a stent delivery system 100 designed and configured for delivery and/or implantation of a stent 130 in a body lumen 10. The term “stent” may be used interchangeably with the term “endoprosthesis” herein. The system 100 may include an elongate tubular member 110 (e.g., an outer tubular member) having a lumen 112 extending therein and/or therethrough. In some alternative embodiments, the elongate tubular member 110 may include a plurality of lumens extending therein and/or therethrough. In at least some embodiments, the system 100 may include an elongate inner member 120 disposed within the lumen 112 of the elongate tubular member 110. In some embodiments, the elongate inner member 120 may be a tubular member having a lumen 122 extending therein and/or therethrough. In some embodiments, the lumen 122 of the elongate inner member 120 may be a guidewire lumen. In some embodiments, the lumen 122 of the elongate inner member 120 may be used for irrigation and/or aspiration. Other configurations are also contemplated. In some alternative embodiments, the elongate inner member 120 may be a solid shaft.

In some embodiments, the elongate inner member 120 may be axially translatable relative to the elongate tubular member 110. In some embodiments, the elongate tubular member 110 may be translatable in a proximal direction relative to the elongate inner member 120. For example, the elongate inner member 120 may be held in a fixed position while the elongate tubular member 110 is withdrawn proximally. In some embodiments, the elongate tubular member 110 and the elongate inner member 120 may be configured to be advanced to a target site together and/or simultaneously. In at least some embodiments, during advancement to the target site, the elongate tubular member 110 and the elongate inner member 120 may be disposed and/or held in an axially fixed relationship relative to each other. Other configurations are also contemplated.

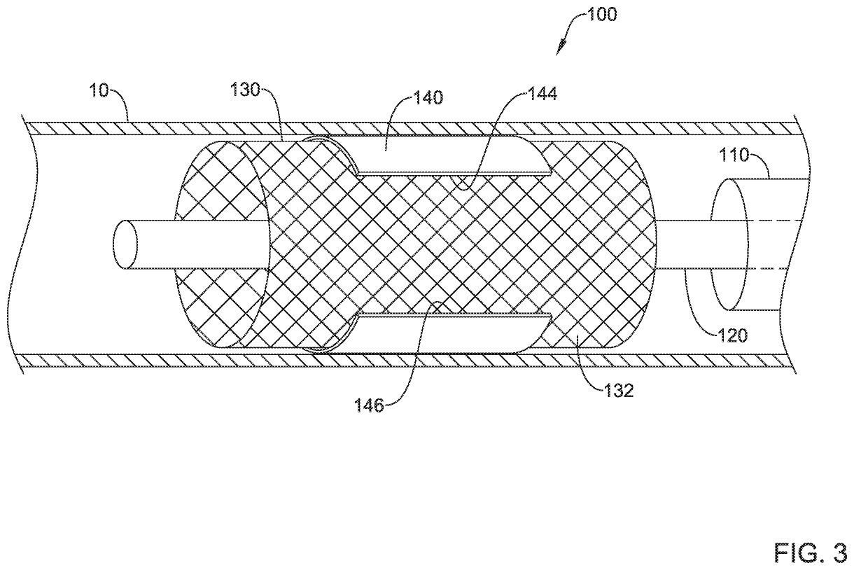

In some embodiments, the system 100 may include a stent 130. The stent 130 may comprise a tubular scaffold extending along a central longitudinal axis and defining a length from a proximal end to a distal end. The stent 130 and/or the tubular scaffold may be configured to shift between a delivery configuration (e.g., a radially collapsed configuration, seen in FIG. 1) and a deployed configuration (e.g., a radially expanded configuration, seen in FIG. 3). The delivery configuration may be a configuration in which the stent 130 is axially elongated and/or radially collapsed or compressed compared to the deployed configuration. The deployed configuration may be a configuration in which the stent 130 is axially shortened and/or radially expanded compared to the delivery configuration.

In some embodiments, the stent 130 may be disposed within the lumen 112 of the elongate tubular member 110 in the delivery configuration. In some embodiments, the elongate tubular member 110 may constrain the stent 130 in the delivery configuration when the stent 130 is disposed within the lumen 112 of the elongate tubular member 110. In some embodiments, the stent 130 may be disposed radially between the elongate tubular member 110 and the elongate inner member 120. In some embodiments, the stent 130 may be attached to, crimped onto, and/or otherwise retained by the elongate inner member 120. Other configurations are also contemplated.

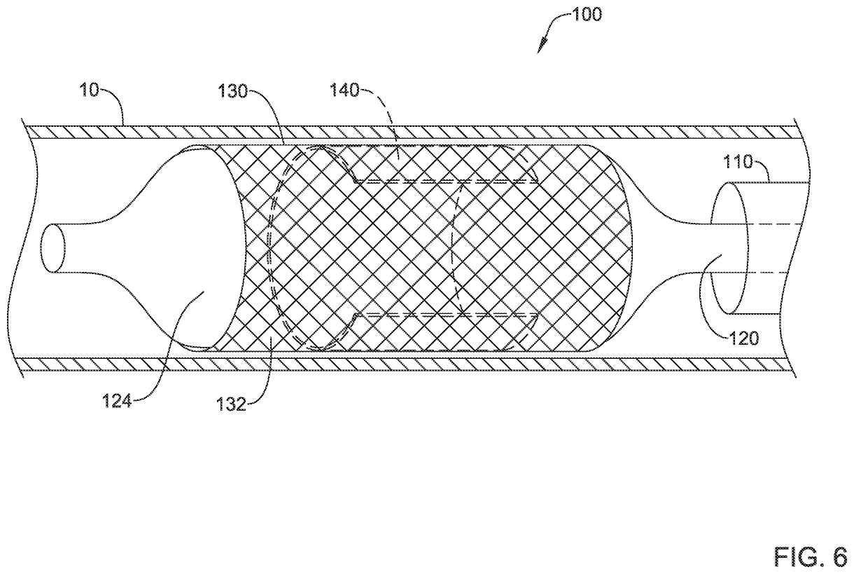

In some embodiments, the stent 130 and/or the tubular scaffold may be self-expandable. For example, the stent 130 and/or the tubular scaffold may be formed from a superelastic and/or shape memory material. In some embodiments, the stent 130 and/or the tubular scaffold may be mechanically expandable. In some embodiments, the elongate inner member 120 may include an expandable member 124 (e.g., FIG. 6), such as an inflatable balloon, a mechanical expansion member, an actuation member, etc., configured to shift the stent 130 and/or the tubular scaffold from the delivery configuration to the deployed configuration.

During delivery to a treatment site, the stent 130 and/or the tubular scaffold may be disposed within the lumen 112 of the elongate tubular member 110 in the delivery configuration. Upon removal and/or release from the lumen 112 of the elongate tubular member 110, the stent 130 and/or the tubular scaffold may shift and/or may be shifted from the delivery configuration to the deployed configuration.

The stent 130 and/or the tubular scaffold may include and/or be formed with a plurality of cells. In some embodiments, the stent 130 and/or the tubular scaffold may include and/or be formed from one or more filaments interwoven around the central longitudinal axis of the stent 130 and/or the tubular scaffold. In at least some embodiments, the one or more filaments may form and/or define the plurality of cells. In some embodiments, the stent 130 and/or the tubular scaffold may be braided, knitted, or woven from the one or more filaments. In some embodiments, the one or more filaments may include wire(s), thread(s), strand(s), etc. In some embodiments, the one or more filaments may define interstices 132 (e.g., openings) formed in and/or through a wall of the stent 130 and/or the tubular scaffold. Alternatively, in some embodiments, the stent 130 and/or the tubular scaffold may be a monolithic structure formed from a cylindrical tubular member, such as a single, cylindrical laser-cut tubular member, in which the remaining (e.g., unremoved) portions of the tubular member form the stent 130 and/or the tubular scaffold with interstices 132 formed in and/or through the wall of the stent 130 and/or tubular scaffold. In yet another alternative, the stent 130 and/or the tubular scaffold may be formed from a flat sheet of material having interstices 132 formed therein that is then rolled and fixed into a tubular shape. Other configurations are also contemplated.

The stent 130 and/or the tubular scaffold may be substantially tubular and/or may include a lumen extending axially therethrough along the central longitudinal axis of the stent 130 and/or the tubular scaffold. In some embodiments, the stent 130 and/or the tubular scaffold may have an axial length of about 20 millimeters to about 250 millimeters, about 40 millimeters to about 225 millimeters, about 60 millimeters to about 200 millimeters, about 80 millimeters to about 175 millimeters, about 100 millimeters to about 150 millimeters, or another suitable range. In some embodiments, the stent 130 and/or the tubular scaffold may have a radial outer dimension or radial extent of about 4 millimeters to about 30 millimeters, about 6 millimeters to about 25 millimeters, about 8 millimeters to about 20 millimeters, about 10 millimeters to about 15 millimeters, or another suitable range. Other configurations are also contemplated. Some suitable but non-limiting materials for the stent 130, the tubular scaffold, and/or components or elements thereof, for example metallic materials and/or polymeric materials, are described below.

In some embodiments, the stent 130 and/or the tubular scaffold may include a polymeric covering extending along the tubular scaffold. In some embodiments, the polymeric covering may be fixedly secured to, bonded to, or otherwise attached along and/or about the circumference of the tubular scaffold. In some embodiments, in at least some locations where the polymeric covering touches the tubular scaffold, the polymeric covering may be fixedly secured to, bonded to, or otherwise attached to the tubular scaffold. In some embodiments, the polymeric covering may be fixedly secured to, bonded to, or otherwise attached to the tubular scaffold at each and every location where the polymeric covering touches the tubular scaffold. In some embodiments, the tubular scaffold, or portions thereof, may be embedded within the polymeric covering. Other configurations are also contemplated.

In some embodiments, the polymeric covering may extend along an entire length of the stent 130 and/or the tubular scaffold. In some embodiments, the polymeric covering may extend along a portion of the length of the stent 130 and/or the tubular scaffold. In some embodiments, the polymeric covering may extend discontinuously between the proximal end of the stent 130 and/or the tubular scaffold and the distal end of the stent 130 and/or the tubular scaffold. In some embodiments, the polymeric covering may extend continuously from the proximal end of the stent 130 and/or the tubular scaffold to the distal end of the stent 130 and/or the tubular scaffold. Other configurations are also contemplated. Some suitable examples of materials for the polymeric covering, including but not limited to PTFE, silicone, and the like, are discussed below.

In some embodiments, the system 100 may include an adhesive structure 140 configured to secure the stent 130 and/or the tubular scaffold to the body lumen 10 in the deployed configuration. In some embodiments, the adhesive structure 140 may be formed from a polymeric material. In some embodiments, the adhesive structure 140 may include a cross-linked polymer. In some embodiments, the adhesive structure 140 may include a plurality of interwoven fibers 142 (e.g., detail of FIG. 1), in some instances forming a fibrous patch. In some embodiments, the plurality of interwoven fibers 142 may include and/or be formed from a polymeric material or a plurality of polymeric materials. In some embodiments, the interwoven fibers of the adhesive structure 140 may have a braided, woven, knit, cross-knit, randomly oriented, or porous construction. In some embodiments, the adhesive structure 140 may include perforations, pores, and/or a plurality of tiny openings disposed therein.

The elongate inner member 120 may be disposed radially inward of the elongate tubular member 110 within the lumen 112 of the elongate tubular member 110 to define an annular space 114. The adhesive structure 140 may be disposed within the lumen 112 of the elongate tubular member 110 adjacent the stent 130 and/or the tubular scaffold in a substantially inert state. In at least some embodiments, the stent 130 and the adhesive structure 140 may be disposed within the annular space 114 defined by the elongate inner member 120 and the elongate tubular member 110 in the delivery configuration and/or in the substantially inert state.

In some embodiments, the adhesive structure 140, which may take the form of a fibrous patch, may be unsecured to the stent 130 and/or the tubular scaffold within the lumen 112 of the elongate tubular member 110, within the annular space 114, and/or in the substantially inert state. In some embodiments, the adhesive structure 140 may be detached from the stent 130 and/or the tubular scaffold within the lumen 112 of the elongate tubular member 110, within the annular space 114, and/or in the substantially inert state. As referred to herein, being unsecured or detached from the stent 130 means that although the adhesive structure 140 is in contact with the stent 130, there is no bond or connection between adhesive structure 140 and the stent 130 while placed against the stent 130 in its inert state. In some embodiments, the adhesive structure 140 may extend circumferentially around a majority of a circumference of the stent 130 and/or the tubular scaffold in the delivery configuration and/or within the annular space 114. In some embodiments, the adhesive structure 140 may extend longitudinally along the length of the stent 130 and/or the tubular scaffold. In some embodiments, the adhesive structure 140 may extend longitudinally along a majority of the length of the stent 130 and/or the tubular scaffold. In some embodiments, the adhesive structure 140 may extend longitudinally along the entire length of the stent 130 and/or the tubular scaffold. In some embodiments, in the substantially inert state the adhesive structure 140 may have a thickness between about 0.100 millimeters (mm) and about 2.750 mm, between about 0.150 mm and about 2.500 mm, between about 0.200 mm and about 2.250 mm, between about 0.250 mm and about 2.000 mm, between about 0.500 mm and about 1.750 mm, between about 0.750 mm and about 1.500 mm, etc.

In some embodiments, the adhesive structure 140 may be configured to adhere to the stent 130 and/or the tubular scaffold and the body lumen 10 after the stent 130 and/or the tubular scaffold and the adhesive structure 140 are deployed into the body lumen 10 from the lumen 112 of the elongate tubular member 110. In some embodiments, the adhesive structure 140 may include an adhesive element that may be activated upon deployment within the body lumen 10. In some embodiments, the adhesive element may extend continuously along the adhesive structure 140. In some embodiments, the adhesive element may extend continuously along the entire length of the adhesive structure 140. In some embodiments, the adhesive element may extend discontinuously along the adhesive structure 140. Other configurations are also contemplated.

In some embodiments, the adhesive element and/or the adhesive structure 140 may include a bioadhesive material. In some embodiments, the bioadhesive material may include natural polymeric materials, as well as synthetic materials, and synthetic materials formed from biological monomers such as sugars. In some embodiments, the bioadhesives may be obtained from the secretions of microbes or by marine mollusks and crustaceans. In some embodiments, the bioadhesives are designed to adhere to biological tissue. Some examples of bioadhesives may include, but are not limited to, mucoadhesives, amino adhesives, adhesive surface proteins, adhesively modified polymers, catechol moieties, polymer materials, polysaccharides, hydrogels, cross-linked or uncross-linked minigel particles, and so forth, as well as mixtures and/or combinations thereof.

In at least some embodiments, the term “substantially inert state” may include and/or refer to the adhesive structure 140 being non-reactive and/or may exhibit non-adhesive properties with respect to adjacent elements of the system 100 and/or the body lumen 10. For example, in the substantially inert state, the adhesive structure 140 may not adhere to the stent 130, the elongate inner member 120, and/or the elongate tubular member 110. Additionally, in the substantially inert state, the adhesive structure 140 may not adhere to the body lumen 10. In some embodiments, in the substantially inert state, the adhesive structure 140 may be substantially dry, dehydrated, or devoid of moisture. In some embodiments, adding or introducing fluid(s), moisture, and/or a selected type of fluid to the adhesive structure 140 may activate the adhesive element associated with the adhesive structure 140. In situations and/or configurations where the adhesive structure 140 and/or the adhesive element is “sticky”, “tacky”, etc. with respect to adjacent structures and/or elements, the adhesive structure 140 may be assumed to no longer be in the substantially inert state.

In some embodiments, the adhesive structure 140 may include a first longitudinal edge 144 and a second longitudinal edge 146 opposite the first longitudinal edge 144. The first longitudinal edge 144 and the second longitudinal edge 146 may be oriented substantially parallel to the central longitudinal axis of the stent 130 and/or the tubular scaffold. In the delivery configuration and/or when the adhesive structure 140 is disposed within the lumen 112 of the elongate tubular member 110 and/or within the annular space 114, the first longitudinal edge 144 may be disposed adjacent to the second longitudinal edge 146. In at least some embodiments, in the delivery configuration and/or when the adhesive structure 140 is disposed within the lumen 112 of the elongate tubular member 110 and/or within the annular space 114, the first longitudinal edge 144 may extend generally parallel to and be spaced apart from the second longitudinal edge 146 to define a longitudinal gap 148 between the first longitudinal edge 144 and the second longitudinal edge 146. In some embodiments, the adhesive structure 140 may be devoid of circumferential overlap (e.g., in a circumferential arc around the central longitudinal axis of the stent 130) between the first longitudinal edge 144 and the second longitudinal edge 146. Circumferential overlap between the first longitudinal edge 144 and the second longitudinal edge 146 within the lumen 112 of the elongate tubular member 110 and/or within the annular space 114 may undesirably cause pinching and/or binding of the stent 130 and the adhesive structure 140 within and/or against the elongate inner member 120 and the elongate tubular member 110, thereby rendering deployment of the stent 130 and the adhesive structure 140 more difficult and/or increasing the chance(s) of damage to the system 100 and/or elements thereof.

In some embodiments, in the deployed configuration and/or when the adhesive structure 140 is disposed within the body lumen 10, the first longitudinal edge 144 and the second longitudinal edge 146 may be spaced farther apart (e.g., the longitudinal gap 148 may be wider) than in the delivery configuration and/or when the adhesive structure 140 is disposed within the lumen 112 of the elongate tubular member 110 and/or within the annular space 114.

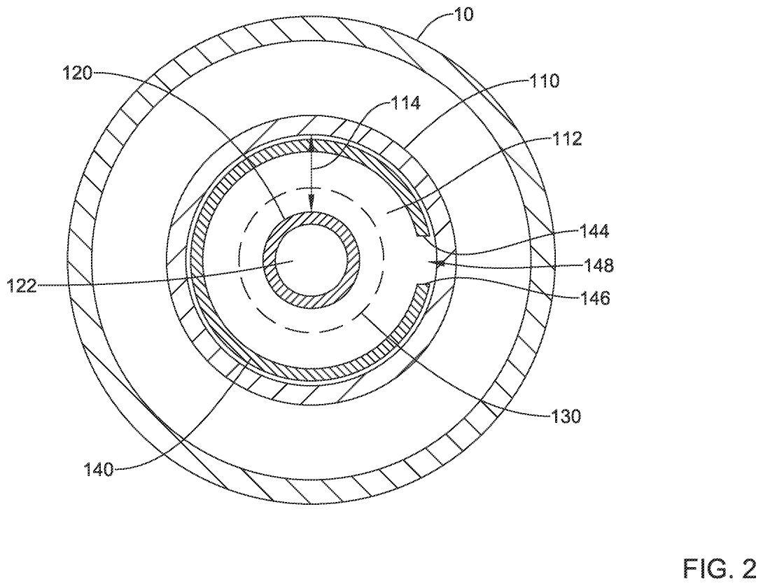

In some embodiments, wetting the adhesive structure 140 with a fluid may activate the adhesive element. In other instances, the adhesive element may be activated by another stimulus. In at least some embodiments, the adhesive element may be pressure sensitive. In some embodiments, the adhesive structure 140 may be disposed radially outward of the stent 130 and/or the tubular scaffold within the lumen 112 of the elongate tubular member 110, as seen in FIGS. 1-2. In some embodiments, an entirety of the adhesive structure 140 may be disposed radially outward of the stent 130 and/or the tubular scaffold within the lumen 112 of the elongate tubular member 110. In some embodiments, after deployment of the stent 130 and the adhesive structure 140 in the body lumen 10, the adhesive structure 140 may be disposed substantially radially outward of the stent 130 and/or the tubular scaffold, as seen in FIG. 3. In some embodiments, after deployment of the stent 130 and the adhesive structure 140 in the body lumen 10, the entirety of the adhesive structure 140 may be disposed radially outward of the stent 130 and/or the tubular scaffold. Other configurations are also contemplated. Thus the radially outward expanding force exerted on the adhesive structure 140 by the stent 130 may activate the pressure sensitive adhesive.

In some embodiments, a method of securing the stent 130 within the body lumen 10 may include exposing the adhesive structure 140 to a target site within the body lumen 10 and/or deploying the adhesive structure 140 at the target site within the body lumen 10. In some embodiments, the system 100 may include a fluid source which may be in fluid communication with the lumen 112 of the elongate tubular member 110 and/or the annular space 114 between the elongate tubular member 110 and the elongate inner member 120. In some embodiments, a fluid may be injected into the annular space 114 and/or the longitudinal gap 148 as the adhesive structure 140 is being exposed to the target site and/or as the adhesive structure 140 is deployed from the lumen 112 of the elongate tubular member 110. In some embodiments, the fluid (e.g., saline) injected into the annular space 114 and/or the longitudinal gap 148 may wet the adhesive structure 140 and/or activate the adhesive element as the adhesive structure 140 is being exposed to the target site within the body lumen 10 and/or as the adhesive structure 140 is deployed from the lumen 112 of the elongate tubular member 110. In some embodiments, fluids (e.g., bodily fluids, blood, mucous, etc.) present within the body lumen 10 may wet the adhesive structure 140 or may aid in wetting the adhesive structure 140.

As the stent 130 may be tightly held in a radially contracted state within the elongate tubular member 110, the presence of the longitudinal gap 148 extending along the length of the adhesive structure 140 may permit the fluid to flow along the longitudinal gap 148 to reach the distal end of the adhesive structure 140 while the stent 130 and the adhesive structure are constrained within the lumen of the elongate tubular member 110.

In some embodiments, the method of securing the stent 130 within the body lumen 10 may include deploying the stent 130 at the target site within the body lumen 10. In some embodiments, after the stent 130 and/or the tubular scaffold and the adhesive structure 140 are deployed into the body lumen 10, the stent 130 may be shifted to the deployed configuration such that the stent 130 and/or the tubular scaffold exerts a radially outward force against the adhesive structure 140 and the body lumen 10, as seen in FIG. 3. In some embodiments, deploying the stent 130 at the target site within the body lumen 10 and/or shifting the stent 130 to the deployed configuration may move the adhesive structure 140 into contact with the target site and/or the body lumen 10. In some embodiments, the method of securing the stent 130 within the body lumen 10 may include applying radially outward force to the adhesive structure 140 for a predetermined period of time sufficient to adhere the adhesive structure to the stent 130 and the target site (e.g., the body lumen 10). In some embodiments, after the stent 130 and the adhesive structure 140 are deployed into the body lumen 10 from the lumen 112 of the elongate tubular member 110, the adhesive structure 140 may be configured to adhere to an outer surface of the stent 130 and/or the adhesive structure 140 may be configured to adhere to the body lumen 10.

In some embodiments, the radially outward force may increase adhesion between the stent 130 and/or the tubular scaffold and the adhesive structure 140 and the radially outward force may increase adhesion between the adhesive structure 140 and the body lumen 10. In some embodiments, the radially outward force may be applied to the adhesive structure 140 and/or the body lumen 10 for the predetermined period of time to permit the adhesive element to set up and/or to permit cross-linkage to occur between the adhesive structure 140 and the adhesive element. In some embodiments, after the adhesive element sets up and/or cross-linkage occurs between the adhesive structure 140 and the adhesive element, the adhesive structure 140 may be permanently adhered to the stent 130 and/or the target site within the body lumen 10.

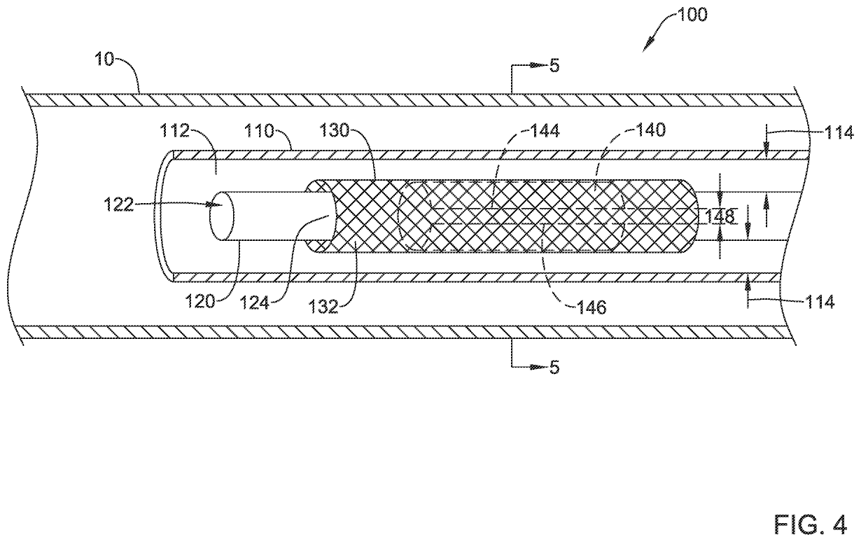

FIGS. 4-6 illustrate selected aspects of an alternative configuration of the system 100 described herein. Except as expressly described herein with respect to FIGS. 4-6, the system 100 may be configured, may be constructed, and/or may function as described above with respect to FIGS. 1-3.

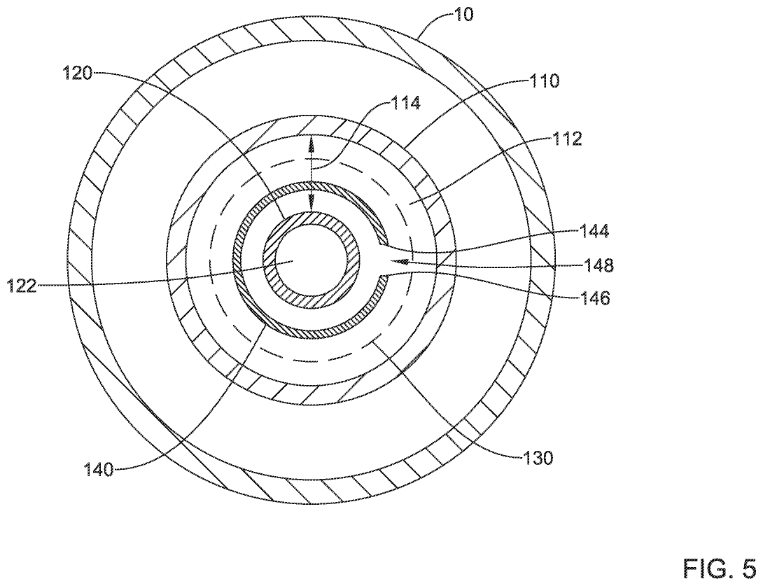

In some embodiments, the elongate inner member 120 may include an expandable member 124. The stent 130 and/or the adhesive structure 140 may be disposed radially outward of the elongate inner member 120 and the expandable member 124. In some embodiments, the stent 130 and/or the adhesive structure 140 may be disposed directly over the expandable member 124. The adhesive structure 140 may be disposed radially inward of the stent 130 and/or the tubular scaffold within the lumen 112 of the elongate tubular member 110 and/or the adhesive structure 140 may be disposed radially inward of the stent 130 and/or the tubular scaffold within the annular space 114 defined by the elongate inner member 120 and the elongate tubular member 110, as seen in FIGS. 4-5.

In some embodiments, after exposing the adhesive structure 140 to the target site and/or the body lumen 10, the method of securing the stent 130 within the body lumen 10 may include deploying the stent 130 at the target site to move the adhesive structure 140 into contact with the target site and/or the body lumen 10. In some embodiments, the expandable member 124, when shifted from a delivery configuration to an expanded configuration, may be configured to move the adhesive structure 140 into contact with an inner surface of the stent 130 and/or the tubular scaffold, as seen in FIG. 6. In some embodiments, the expandable member 124, when shifted from the delivery configuration to the expanded configuration, may be configured to move the adhesive structure 140 into contact with the target site and/or the body lumen 10.

In some embodiments, after deployment of the stent 130 and the adhesive structure 140 in the body lumen 10, the adhesive structure 140 may be disposed substantially radially inward of the stent 130 and/or the tubular scaffold. In some embodiments, after deployment of the stent 130 and the adhesive structure 140 in the body lumen 10, and after the stent 130 has been shifted to the deployed configuration, at least some of the adhesive structure 140 may protrude into and/or through the interstices 132 formed in the wall of the stent 130 and/or the tubular scaffold. In some embodiments, after the stent 130 has been shifted to the deployed configuration, and after the expandable member 124 has been shifted to the expanded configuration, at least some of the adhesive structure 140 may protrude into and/or through the interstices 132 formed in the wall of the stent 130 and/or the tubular scaffold and into contact with the body lumen 10.

In some embodiments, the adhesive structure 140 may be configured to adhere to the stent 130 and/or the tubular scaffold and the body lumen 10 after the stent 130 and/or the tubular scaffold and the adhesive structure 140 are deployed into the body lumen 10 from the lumen 112 of the elongate tubular member 110. In some embodiments, after the stent 130 and the adhesive structure 140 are deployed into the body lumen 10 from the lumen 112 of the elongate tubular member 110, the adhesive structure 140 may be configured to adhere to an inner surface of the stent 130 and the adhesive structure 140 may be configured to adhere to the body lumen 10 through the interstices 132 formed in the wall of the stent 130 and/or the tubular scaffold.

In some instances, the expandable member 124 (e.g., an inflatable balloon), any include a plurality of pores or apertures permitting fluid from inside the expandable member 124 to weep out through the sidewall of the expandable member 124 into direct contact with the adhesive structure 140 to wet the adhesive structure 140 during deployment, thus activating the adhesive element of the adhesive structure 140.

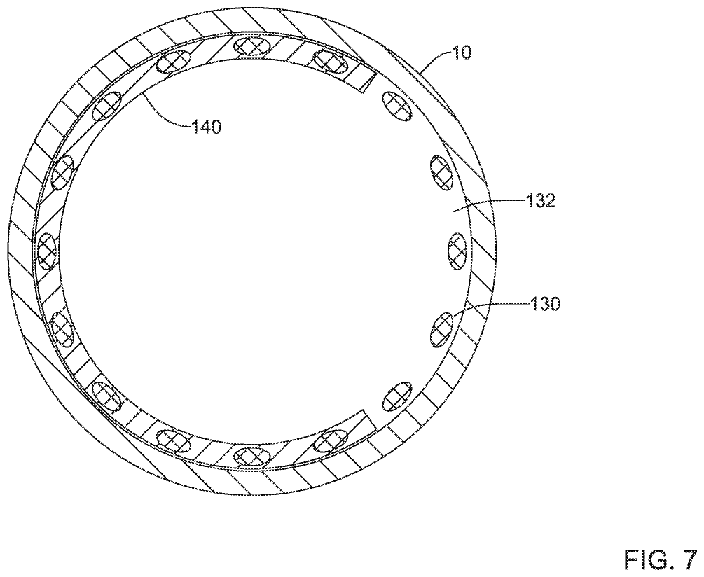

FIG. 7 illustrates selected aspects of the stent 130 and/or the adhesive structure 140 following deployment within the body lumen 10. At least some aspects seen in FIG. 7 may occur and/or may be present in the embodiment(s) of FIGS. 1-6. In some embodiments, applying radially outward force to the adhesive structure 140 and/or the stent 130, as discussed herein, at least partially embeds the stent 130 and/or the tubular scaffold within the adhesive structure 140. In some embodiments, wetting the adhesive structure 140 may soften the adhesive structure 140. In some embodiments, after wetting the adhesive structure 140 and/or after applying the radially outward force to the adhesive structure 140 and/or the stent 130 of FIGS. 1-3, the stent 130 and/or the tubular scaffold may be pushed and/or urged into the adhesive structure 140 to at least partially embed the stent 130 and/or the tubular scaffold within the adhesive structure 140. In some embodiments, after wetting the adhesive structure 140 and/or after applying the radially outward force to the adhesive structure 140 and/or the stent 130 of FIGS. 4-6, the adhesive structure 140 may be pushed through and/or around the stent 130 and/or the tubular scaffold to at least partially embed the stent 130 and/or the tubular scaffold within the adhesive structure 140. Other configurations are also contemplated.

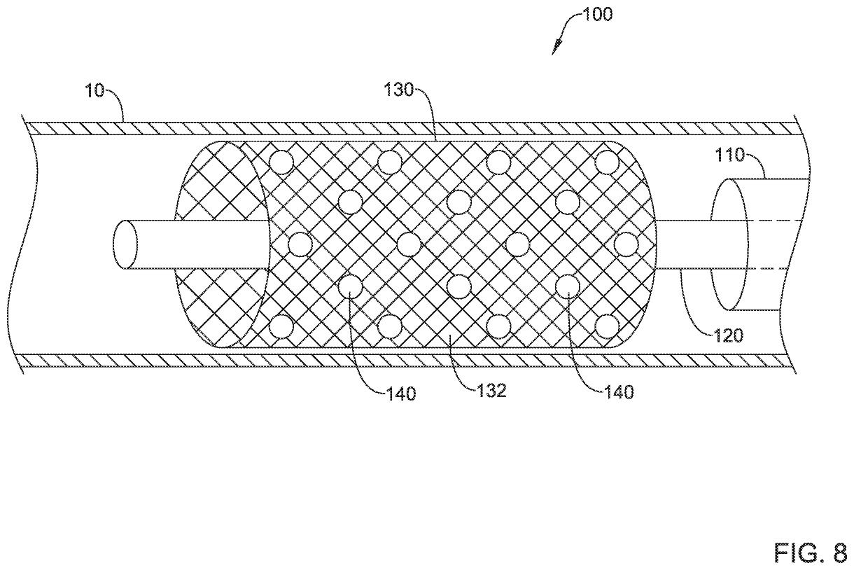

FIG. 8 illustrates selected aspects of an alternative configuration of the system 100. In some embodiments, the adhesive structure 140 may comprise and/or may include a plurality of discrete regions disposed along the length of the stent 130 and/or the tubular scaffold. In some embodiments, the plurality of discrete regions may be spaced apart from one another along the length of the stent 130. In some embodiments, the plurality of discrete regions may be regularly spaced apart along the length of the stent 130 (e.g., disposed in a defined pattern). In some embodiments, the plurality of discrete regions may be irregularly spaced apart along the length of the stent 130 (e.g., disposed randomly). In some embodiments, the plurality of discrete regions may be disposed along and/or on an outer surface of the stent 130, similar to the embodiment(s) shown in FIGS. 1-3. In some embodiments, the plurality of discrete regions may be disposed along and/or against the inner surface of the stent 130, similar to the embodiment(s) shown in FIGS. 4-6. Other configurations, including various combinations thereof, are also contemplated.

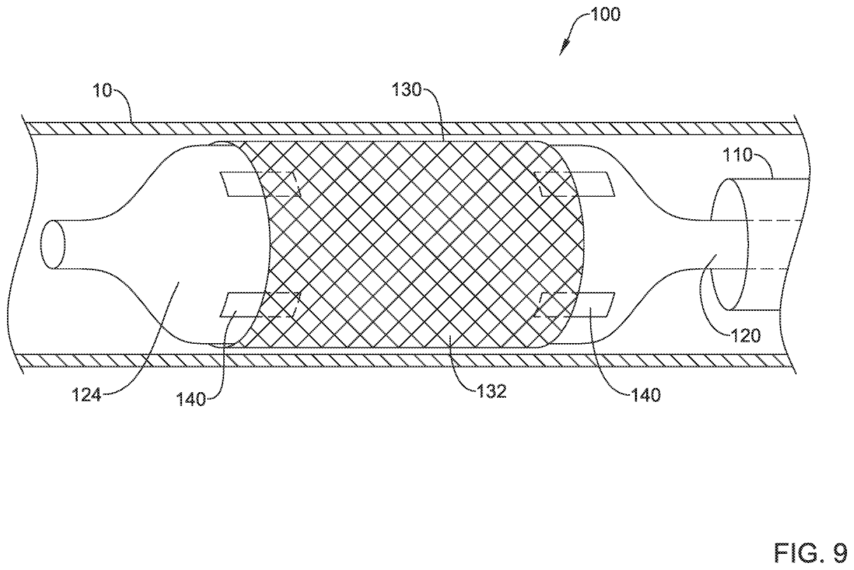

FIG. 9 illustrates selected aspects of an alternative configuration of the system 100. In some embodiments, the adhesive structure 140 may extend longitudinally beyond at least one end of the stent 130. For example, the adhesive structure 140 may be positioned at a distal end of the stent 130 and may extend distally beyond the distal end of the stent 130. Additionally or alternatively, the adhesive structure 140 may be positioned at a proximal end of the stent and extend proximally beyond the proximal end of the stent 130. In some instances the adhesive structure 140 extending beyond the proximal and/or distal end of the stent 130 may be a cuff extending entirely around the circumference of the end of the stent 130. In some embodiments, the adhesive structure 140 may comprise and/or may include a plurality of discrete regions or strips disposed at and/or immediately adjacent to at least one end of the stent 130 and/or the tubular scaffold. In some embodiments, the plurality of discrete regions or strips may be circumferentially spaced apart about the circumference of the stent 130. In some embodiments, the plurality of discrete regions or strips may be regularly spaced apart about the circumference of the stent 130 (e.g., disposed in a defined pattern). In some embodiments, the plurality of discrete regions or strips may be irregularly spaced apart about the circumference of the stent 130 (e.g., disposed randomly). In some embodiments, the plurality of discrete regions or strips may be disposed along and/or on an outer surface of the stent 130, similar to the embodiment(s) shown in FIGS. 1-3. In some embodiments, the plurality of discrete regions or strips may be disposed along and/or against the inner surface of the stent 130, similar to the embodiment(s) shown in FIGS. 4-6. Other configurations, including various combinations thereof, are also contemplated. In one example, an expandable member 124 of the elongate inner member 120 may be configured to apply radially outward force to the adhesive structure 140 (e.g., the plurality of discrete regions) and/or the stent 130 to move the adhesive structure 140 (e.g., the plurality of discrete regions) and/or the stent 130 into contact with the body lumen 10.

The materials that can be used for the various components of the system and the various elements thereof disclosed herein may include those commonly associated with medical devices. For simplicity purposes, the following discussion refers to the system. However, this is not intended to limit the system, devices, and/or methods described herein, as the discussion may be applied to other elements, members, components, or devices disclosed herein, such as, but not limited to, the elongate tubular member, the elongate inner member, the stent, the tubular scaffold, the polymeric cover, the adhesive structure, and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane (for example, Polyurethane 85A), polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, a linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A system for implantation of a stent in a body lumen, comprising:
an elongate tubular member having a lumen extending therein;
a stent configured to shift from a delivery configuration to a deployed configuration; and
an adhesive structure configured to secure the stent to the body lumen in the deployed configuration;
wherein the stent is disposed within the lumen of the elongate tubular member in the delivery configuration;
wherein the adhesive structure is disposed within the lumen of the elongate tubular member adjacent the stent in a substantially inert state;
wherein the adhesive structure is configured to adhere to the stent and the body lumen after the stent and the adhesive structure are deployed into the body lumen from the lumen of the elongate tubular member.

2. The system of claim 1, wherein the adhesive structure is unsecured to the stent within the lumen of the elongate tubular member.

3. The system of claim 1, wherein the adhesive structure includes an adhesive element that is activated upon deployment within the body lumen.

4. The system of claim 3, wherein the adhesive element is pressure sensitive.

5. The system of claim 1, wherein the adhesive structure is disposed radially outward of the stent within the lumen of the elongate tubular member.

6. The system of claim 1, wherein after the stent and the adhesive structure are deployed into the body lumen, the stent exerts a radially outward force against the adhesive structure and the body lumen.

7. The system of claim 6, wherein the radially outward force increases adhesion between the stent and the adhesive structure and between the adhesive structure and the body lumen.

8. The system of claim 1, wherein the adhesive structure extends circumferentially around a majority of a circumference of the stent in the delivery configuration.

9. The system of claim 1, wherein in the substantially inert state the adhesive structure has a thickness between about 0.254 mm and about 2.032 mm.

10. A system for implantation of a stent in a body lumen, comprising:
an elongate tubular member having a lumen extending therein;
a stent configured to shift from a delivery configuration to a deployed configuration; and
an adhesive structure configured to secure the stent to the body lumen in the deployed configuration;
wherein the stent is disposed within the lumen of the elongate tubular member in the delivery configuration;
wherein the adhesive structure is disposed within the lumen of the elongate tubular member adjacent the stent in a substantially inert state;
wherein the adhesive structure is configured to adhere to the stent and the body lumen after the stent and the adhesive structure are deployed into the body lumen from the lumen of the elongate tubular member;
wherein the adhesive structure is disposed radially inward of the stent within the lumen of the elongate tubular member.

11. The system of claim 10, wherein after the stent and the adhesive structure are deployed into the body lumen from the lumen of the elongate tubular member, the adhesive structure is configured to adhere to an inner surface of the stent and the adhesive structure is configured to adhere to the body lumen through interstices formed in the stent.

12. The system of claim 10, wherein the adhesive structure includes a cross-linked polymer.

13. The system of claim 10, wherein the adhesive structure includes a plurality of interwoven fibers defining a patch.

14. The system of claim 10, wherein the adhesive structure extends longitudinally beyond at least one end of the stent.

15. A method of securing a stent within a body lumen, comprising:
advancing an elongate tubular member to a target site within the body lumen, the elongate tubular member having a stent and an adhesive structure retained therein;
exposing the adhesive structure to the target site;
deploying the stent at the target site to move the adhesive structure into contact with the target site; and
applying radially outward force to the adhesive structure for a predetermined period of time sufficient to adhere the adhesive structure to the stent and the target site.

16. The method of claim 15, wherein an elongate inner member is disposed radially inward of the elongate tubular member to define an annular space, the stent and the adhesive structure being disposed within the annular space.

17. The method of claim 16, further comprising injecting a fluid into the annular space as the adhesive structure is being exposed to the target site to wet the adhesive structure.

18. The method of claim 17, wherein the adhesive structure includes an adhesive element and wetting the adhesive structure activates the adhesive element.

19. The method of claim 15, wherein applying radially outward force to the adhesive structure at least partially embeds the stent within the adhesive structure.

20. The method of claim 15, wherein the adhesive structure is detached from the stent within the elongate tubular member.
